(19) United States
(12) Patent Application Publication
(10) Pub. No.: US 2021/0076991 A1
(43) Pub. Date: Mar. 18, 2021

(54) IN-SITU SWEAT RATE MONITORING FOR NORMALIZATION OF SWEAT ANALYTE CONCENTRATIONS

(71) Applicant: THE REGENTS OF THE UNIVERSITY OF CALIFORNIA, Oakland, CA (US)

(72) Inventor: Sam EMAMINEJAD, Los Angeles, CA (US)

(73) Assignee: THE REGENTS OF THE UNIVERSITY OF CALIFORNIA, Oakland, CA (US)

(21) Appl. No.: 16/962,479

(22) PCT Filed: Jan. 15, 2019
(86) PCT No.: PCT/US2019/013647

§ 371 Date: Jul. 15, 2020

Related U.S. Application Data
(60) Provisional application No. 62/617,934, filed on Jan. 16, 2018.

(51) Int. Cl. A61B 5/145; B01L 3/00; A61B 5/1495; A61B 5/1477; A61B 10/00

(52) U.S. Cl. CPC A61B 5/14521; B01L 3/502715; A61B 5/1495; A61B 5/0537; A61B 10/0064; B01L 2300/0645; A61B 5/1477

(57) ABSTRACT

A device for sweat analysis includes: (1) a sensing module configured to induce sweat and generate a sensing signal responsive to a sweat concentration of a target analyte in induced sweat, the sensing module including a calibrating sensor to generate a calibration signal responsive to a secretion rate of the induced sweat; and (2) a processor connected to the sensing module, the processor configured to derive a measurement of the sweat concentration of the target analyte from the sensing signal, and to derive a normalized measurement of a blood concentration of the target analyte from the calibration signal.

### CROSS-REFERENCE TO RELATED APPLICATION

This application claims the benefit of U.S. Provisional Application No. 62/617,934, filed Jan. 16, 2018, the contents of which are incorporated herein by reference in their entirety.

### TECHNICAL FIELD

This disclosure generally relates to devices for sweat analysis.

### BACKGROUND

Despite being a rich source of biomarkers, sweat analysis has not widely used in physiological and clinical settings. This is due to the lack of a suitable methodology to overcome the barriers in correlating sweat readings of an analyte to blood concentrations of the analyte and inferring physiologically meaningful information from sweat. Attempts have been made in demonstrating some level of correlation between blood and sweat concentrations in the context of certain biomarkers. However, correlations can vary for each analyte, can differ from subject to subject, and can be inconsistent during an entire period of evaluation. These discrepancies are primarily attributed to variations in the sweat-gland secretion rate—the major operational factor in the sweat secretion process. Attempts towards implementing sweat sensors have demonstrated the ability to perform in-situ sweat measurements. However, due to the lack of a suitable methodology to mitigate the dependency of sweat readings on secretion rate, the measurements provided limited physiological insight.

It is against this background that a need arose to develop the embodiments described herein.

### SUMMARY

In some embodiments, a device for sweat analysis includes: (1) a sensing module configured to induce sweat and generate a sensing signal responsive to a sweat concentration of a target analyte in induced sweat, the sensing module including a calibrating sensor to generate a calibration signal responsive to a secretion rate of the induced sweat; and (2) a processor connected to the sensing module, the processor configured to derive a measurement of the sweat concentration of the target analyte from the sensing signal, and to derive a normalized measurement of a blood concentration of the target analyte from the calibration signal.

In some embodiments, a method for sweat analysis includes: (1) deriving a concentration of a target analyte in sweat; (2) deriving a secretion rate of the sweat; and (3) deriving a concentration of the target analyte in blood from the concentration of the target analyte in the sweat and the secretion rate.

In some embodiments, a non-transitory computer-readable storage medium includes instructions to: (1) derive a concentration of a target analyte in sweat; (2) derive a secretion rate of the sweat; and (3) derive a concentration of the target analyte in blood from the concentration of the target analyte in the sweat and the secretion rate.

Other aspects and embodiments of this disclosure are also contemplated. The foregoing summary and the following detailed description are not meant to restrict this disclosure to any particular embodiment but are merely meant to describe some embodiments of this disclosure.

### BRIEF DESCRIPTION OF THE DRAWINGS

For a better understanding of the nature and objects of some embodiments of this disclosure, reference should be made to the following detailed description taken in conjunction with the accompanying drawings.

FIG. 1 is a schematic illustration of a wearable device for sweat analysis according to some embodiments.

FIG. 2 is a schematic illustration of a sensing compartment C according to some embodiments.

FIG. 3 is a schematic illustration of a microfluidics-based implementation of a secretion rate sensor according to some embodiments.

FIG. 4 is a demonstration of microbubble generation and tracking for sweat rate monitoring according to some embodiments.

### DETAILED DESCRIPTION

To achieve a normalized measure of target analytes, embodiments of this disclosure are directed to a device and a method of in-situ sweat secretion rate monitoring. The secretion rate information allows for characterizing and decoupling the confounding effect of the influential secretion parameters in the transport of the target analytes into sweat. The secretion rate information also can be used to derive a measure of hydration status and temperature and oxygen regulation.

Some embodiments are directed to a wearable device for sweat analysis. In some embodiments, the wearable device includes a sensing module, which includes one or more sensing compartments. Each sensing compartment includes iontophoresis electrodes/hydrogel layer for sweat induction, an array of one or more sweat analyte sensors, and one or more calibrating sensors, including a secretion rate sensor. Through activating the iontophoresis functionality in the sensing compartment, a secretory agonist in a hydrogel layer is delivered to sweat glands of an individual to stimulate sweat secretion. By measuring a secretion rate of the individual using the secretion rate sensor, normalization of sweat analyte measurements can be performed with respect to the measured secretion rate. The sensing module interfaces a wireless circuit board. The circuit board includes integrated circuitry (e.g., one or more chips) and other electronic devices to realize iontophoresis, signal conditioning (e.g., analog/digital signal processing), control (e.g., for setting an iontophoresis current), and wireless communication functionalities, thus providing a fully integrated and programmable platform.

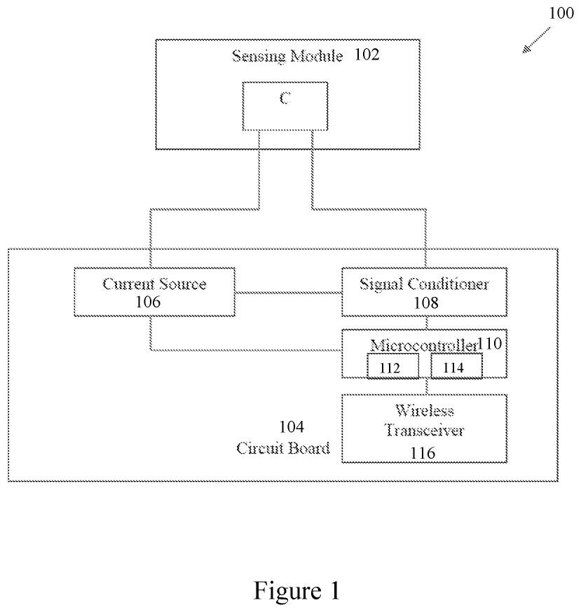

FIG. 1 is a schematic illustration of a wearable device 100 for sweat analysis according to some embodiments. The wearable device 100 includes a sensing module 102 and a circuit board 104, where the sensing module 102 interfaces with the circuit board 104 through electrical connections. The sensing module 102 includes at least one sensing compartment C. Although the one sensing compartment C is shown, in general, one or more sensing compartments can be included in the sensing module 102 and can be integrated on a common substrate.

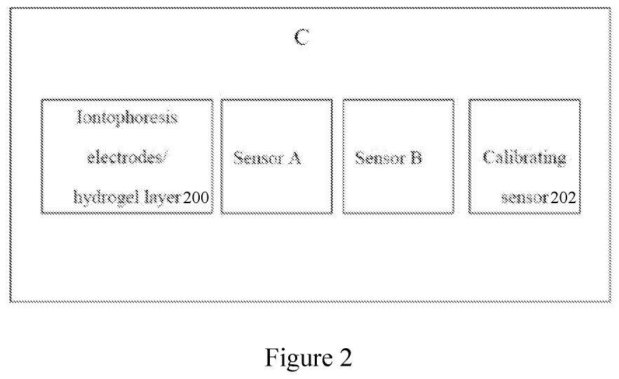

FIG. 2 is a schematic illustration of the sensing compartment C according to some embodiments. Any additional sensing compartments can be similarly implemented as illustrated in FIG. 2. The sensing compartment C includes a pair of iontophoresis electrodes/hydrogel layer 200 for sweat induction, an array of sweat analyte sensors A and B, and a calibrating sensor 202. The hydrogel layer is adjacent to the iontophoresis electrodes, and the iontophoresis electrodes are configured to interface a skin with the hydrogel layer in between. The hydrogel layer includes a secretory agonist (e.g., a cholinergic sweat gland secretory stimulating compound, such as pilocarpine), which is released when an electrical current is applied to the iontophoresis electrodes. Each of sensors A and B includes a sensing layer and a sensing electrode adjacent to the sensing layer. The sensors A and B are configured to sense respective and different analytes, by generating sensing signals responsive to presence or levels of such analytes in induced sweat. For example, analytes can be selected from metabolites, electrolytes, proteins, and heavy metals. For example, the sensors A and B can be different sensors selected from a glucose sensor including an enzyme in a sensing layer (e.g., glucose oxidase), a lactate sensor including an enzyme in a sensing layer (e.g., lactate oxidase), a Na⁺ sensor, a Cl⁻ sensor, and Ca²⁺ sensor. Although the two sensors A and B are illustrated in FIG. 2, in general, one or more sensors can be included in the sensing compartment C. The calibrating sensor 202 is a secretion rate sensor, which generates a calibration signal responsive to a secretion rate of a skin such that responses of the sensors A and B can be adjusted or calibrated according to such calibration signal. The secretion rate sensor can be implemented as, for example, a capacitive humidity sensor, which can include a hydroscopic dielectric material disposed between a pair of electrodes, and where a capacitance of the sensor varies according to an amount of sweat present in the dielectric material. Other suitable implementations of the secretion rate sensor can be used. In particular, in some embodiments, the secretion rate sensor can be implemented as a microfluidic channel, in which a secretion rate can be accurately inferred by measuring a velocity of microbubbles generated from sweat within the microfluidic channel. Further details of such a microfluidics-based implementation of the secretion rate sensor are provided below. Although the one calibrating sensor is illustrated in FIG. 2, one or more additional calibrating sensors can be included in the sensing compartment C, such as a pH sensor or a skin temperature sensor, which generates a calibration signal responsive to a pH or a skin temperature.

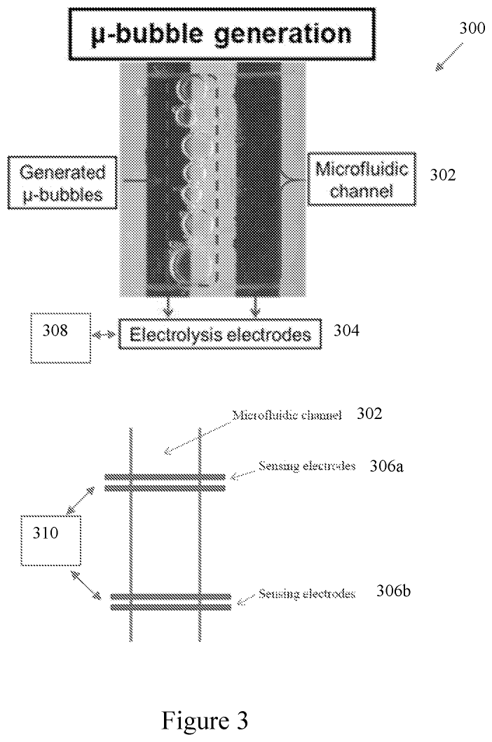

FIG. 3 is a schematic illustration of a microfluidics-based implementation of a secretion rate sensor 300 according to some embodiments. The secretion rate sensor 300 includes a microfluidic channel 302, along with electrolysis electrodes 304 and impedance sensing electrodes 306a and 306b positioned along a flow path of the channel 302. The electrolysis electrodes 304 are positioned across the microfluidic channel 302 in an upstream portion of the channel 302, and are activated (through connection to an electrical source 308) to generate microbubbles from sweat (in a burst mode). The impedance sensing electrodes 306a and 306b are positioned in a downstream portion of the channel 302, and operate to measure a velocity of the generated microbubbles. To facilitate high signal-to-noise measurements, two pairs of impedance sensing electrodes 306a and 306b—which are spaced apart in the downstream portion of the channel 302—are included to measure consecutive changes to a baseline impedance of the channel 302 (measured by each pair 306a or 306b and through connection to impedance detection electronics 310) as the bubbles flow through the channel 302 and pass over the sensing electrodes 306a and 306b. In this manner, the presence of the bubbles is detected at two different time points using the two pairs of impedance sensing electrodes 306a and 306b, and, by deriving a time difference between the two detection time points and given dimensions of the channel 302, a volumetric flow rate can be derived as proportionally related to the channel dimensions divided by the time difference.

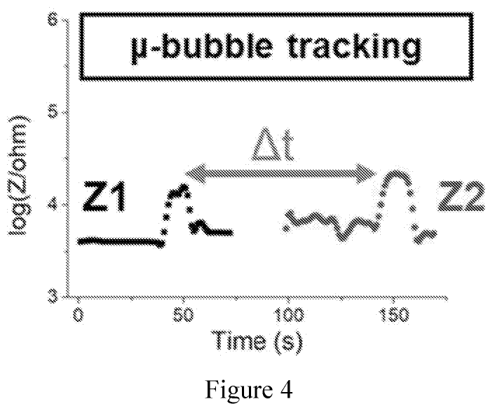

FIG. 4 is a demonstration of microbubble generation and tracking for sweat rate monitoring according to some embodiments. Microbubbles are generated through activation of electrolysis electrodes, and the passage of the microbubbles over two pairs of impedance sensing electrodes results in instantaneous spikes in their respective measured impedance values. A time difference between the spikes can be used to infer a volumetric flow rate.

Referring back to FIG. 1, the circuit board 104 includes a current source 106, which is connected to the sensing compartment C to activate sweat induction. A signal conditioner 108 is also included in the circuit board 104, and can include signal processing circuitry such as one or more analog-to-digital converters, one or more digital-to-analog converters, and one or more filters. A processor 112 and an associated memory 114 storing processor-executable instructions (e.g., included in a microcontroller 110) are also included in the circuit board 104, and are configured to control operation of various components of the sensing module 102 and the circuit board 104. In particular, the processor 112 is configured to direct operation of the sensing compartment C, through control of the current source 106 and the signal conditioner 108. In addition, the processor 112 is configured to adjust or calibrate responses of the sensors A and B according to a calibration signal from the calibrating sensor 202, and to derive analyte measurements according to the calibrated responses. A wireless transceiver 116 is also included in the circuit board 104 to allow wireless communication between the wearable device 100 and an external electronic device, such as a portable electronic device or a remote computing device.

The following further explains operations of normalizing sweat analyte measurements with respect to a measured secretion rate. A concentration of an analyte secreted in sweat can be dependent upon a secretion rate. Since the secretion rate can vary across individuals when subjected to a same or similar sweat induction condition, it is desired to decouple the effect of the secretion rate from a measured concentration of a secreted analyte. For example, a linear model can be used to represent a relationship between a target analyte's concentrations in sweat [M]_{S} and blood [M]_{B} as denoted below:

[M]_{S}=a(Q)[M]_{B}+b(Q)+ε

where Q denotes a secretion rate (which can vary across individuals subjected to a same or similar sweat induction condition), a(Q) and b(Q) are related to secretion accumulation and gland contribution, respectively, and are functions (e.g., linear functions) of the secretion rate Q according to secretion parameters, and ε is a non-secretion parameter capturing a confounding effect. For example, a(Q) can be represented as a₁Q+a₂, and b(Q) can be represented as b₁Q+b₂. By performing a measurement of the secretion rate Q and with given secretion and non-secretion parameters, the effect of the secretion rate Q and its confounding effect can be decoupled from measurements of the target analyte's concentration in sweat to derive normalized measurements of the target analyte that are reflective of blood levels. Although a linear model is explained above, a non-linear model also can be used to represent relationship between the target analyte's concentrations in sweat and blood.

The following are example embodiments of this disclosure.

First Aspect

In some embodiments according to a first aspect, a device for sweat analysis includes: (1) a sensing module configured to induce sweat and generate a sensing signal responsive to a sweat concentration of a target analyte in induced sweat, the sensing module including a calibrating sensor to generate a calibration signal responsive to a secretion rate of the induced sweat; and (2) a processor connected to the sensing module, the processor configured to derive a measurement of the sweat concentration of the target analyte from the sensing signal, and to derive a normalized measurement of a blood concentration of the target analyte from the calibration signal.

In some embodiments, the sensing module includes a pair of iontophoresis electrodes and a secretory agonist-containing hydrogel layer adjacent to the pair of iontophoresis electrodes, and a sweat analyte sensor configured to generate the sensing signal.

In some embodiments, the calibrating sensor includes a humidity sensor.

In some embodiments, the calibrating sensor includes a microfluidic channel, a set of electrolysis electrodes positioned in an upstream portion of the microfluidic channel and configured to generate microbubbles from the induced sweat, and a set of impedance sensing electrodes positioned in a downstream portion of the microfluidic channel and configured to detect the generated microbubbles.

In some embodiments, the set of impedance sensing electrodes includes a first set of impedance sensing electrodes positioned in the downstream portion of the microfluidic channel, and a second set of impedance sensing electrodes positioned in the downstream portion of the microfluidic channel and spaced apart from the first set of impedance sensing electrodes.

In some embodiments, the processor is configured to derive a time difference between two detection time points of the microbubbles at the first set of impedance sensing electrodes and the second set of impedance sensing electrodes, and to derive the secretion rate of the induced sweat based on the time difference.

Second Aspect

In some embodiments according to a second aspect, a method for sweat analysis includes: (1) deriving a concentration of a target analyte in sweat; (2) deriving a secretion rate of the sweat; and (3) deriving a concentration of the target analyte in blood from the concentration of the target analyte in the sweat and the secretion rate.

In some embodiments, deriving the concentration of the target analyte in the blood is performed using a linear model relating the concentration of the target analyte in the sweat to the concentration of the target analyte in the blood.

In some embodiments, deriving the secretion rate of the sweat includes generating microbubbles from the sweat, deriving a time difference between two detection time points of the microbubbles at a first set of impedance sensing electrodes and a second set of impedance sensing electrodes, and deriving the secretion rate of the sweat based on the time difference.

Third Aspect

In some embodiments according to a third aspect, a non-transitory computer-readable storage medium includes instructions to: (1) derive a concentration of a target analyte in sweat; (2) derive a secretion rate of the sweat; and (3) derive a concentration of the target analyte in blood from the concentration of the target analyte in the sweat and the secretion rate.

In some embodiments, the instructions to derive the secretion rate of the sweat include instructions to direct generation of microbubbles from the sweat, derive a time difference between two detection time points of the microbubbles at a first set of impedance sensing electrodes and a second set of impedance sensing electrodes, and derive the secretion rate of the sweat based on the time difference.

As used herein, the singular terms “a,” “an,” and “the” may include plural referents unless the context clearly dictates otherwise. Thus, for example, reference to an object may include multiple objects unless the context clearly dictates otherwise.

As used herein, the term “set” refers to a collection of one or more objects. Thus, for example, a set of objects can include a single object or multiple objects. Objects of a set also can be referred to as members of the set. Objects of a set can be the same or different. In some instances, objects of a set can share one or more common characteristics.

As used herein, the terms “connect,” “connected,” and “connection” refer to an operational coupling or linking. Connected objects can be directly coupled to one another or can be indirectly coupled to one another, such as via one or more other objects.

As used herein, the terms “substantially” and “about” are used to describe and account for small variations. When used in conjunction with an event or circumstance, the terms can refer to instances in which the event or circumstance occurs precisely as well as instances in which the event or circumstance occurs to a close approximation. For example, when used in conjunction with a numerical value, the terms can refer to a range of variation of less than or equal to ±10% of that numerical value, such as less than or equal to ±5%, less than or equal to ±4%, less than or equal to ±3%, less than or equal to ±2%, less than or equal to ±1%, less than or equal to ±0.5%, less than or equal to ±0.1%, or less than or equal to ±0.05%.

Additionally, concentrations, amounts, ratios, and other numerical values are sometimes presented herein in a range format. It is to be understood that such range format is used for convenience and brevity and should be understood flexibly to include numerical values explicitly specified as limits of a range, but also to include all individual numerical values or sub-ranges encompassed within that range as if each numerical value and sub-range is explicitly specified. For example, a range of about 1 to about 200 should be understood to include the explicitly recited limits of about 1 and about 200, but also to include individual values such as about 2, about 3, and about 4, and sub-ranges such as about 10 to about 50, about 20 to about 100, and so forth.

Some embodiments of this disclosure relate to a non-transitory computer-readable storage medium having computer code or instructions thereon for performing various processor-implemented operations. The term “computer-readable storage medium” is used to include any medium that is capable of storing or encoding a sequence of instructions or computer code for performing the operations, methodologies, and techniques described herein. The media and computer code may be those specially designed and constructed for the purposes of the embodiments of the disclosure, or they may be of the kind available to those having skill in the computer software arts. Examples of computer-readable storage media include volatile and non-volatile memory for storing information. Examples of memory include semiconductor memory devices such as erasable programmable read-only memory (EPROM), electrically erasable programmable read-only memory (EEPROM), random-access memory (RAM), and flash memory devices, discs such as internal hard drives, removable hard drives, magneto-optical, compact disc (CD), digital versatile disc (DVD), and Blu-ray discs, memory sticks, and the like. Examples of computer code include machine code, such as produced by a compiler, and files containing higher-level code that are executed by a processor using an interpreter or a compiler. For example, an embodiment of the disclosure may be implemented using Java, C++, or other object-oriented programming language and development tools. Additional examples of computer code include encrypted code and compressed code. Moreover, an embodiment of the disclosure may be downloaded as a computer program product, which may be transferred from a remote computing device via a transmission channel. Another embodiment of the disclosure may be implemented in hardwired circuitry in place of, or in combination with, processor-executable software instructions.

While the disclosure has been described with reference to the specific embodiments thereof, it should be understood by those skilled in the art that various changes may be made and equivalents may be substituted without departing from the true spirit and scope of the disclosure as defined by the appended claims. In addition, many modifications may be made to adapt a particular situation, material, composition of matter, method, operation or operations, to the objective, spirit and scope of the disclosure. All such modifications are intended to be within the scope of the claims appended hereto. In particular, while certain methods may have been described with reference to particular operations performed in a particular order, it will be understood that these operations may be combined, sub-divided, or re-ordered to form an equivalent method without departing from the teachings of the disclosure. Accordingly, unless specifically indicated herein, the order and grouping of the operations are not a limitation of the disclosure.

1. A device for sweat analysis, comprising:
a sensing module configured to induce sweat and generate a sensing signal responsive to a sweat concentration of a target analyte in induced sweat, the sensing module including a calibrating sensor to generate a calibration signal responsive to a secretion rate of the induced sweat; and
a processor connected to the sensing module, the processor configured to derive a measurement of the sweat concentration of the target analyte from the sensing signal, and to derive a normalized measurement of a blood concentration of the target analyte from the calibration signal.

2. The device of claim 1, wherein the sensing module includes:
a pair of iontophoresis electrodes and a secretory agonist-containing hydrogel layer adjacent to the pair of iontophoresis electrodes; and
a sweat analyte sensor configured to generate the sensing signal.

3. The device of claim 1, wherein the calibrating sensor includes a humidity sensor.

4. The device of claim 1, wherein the calibrating sensor includes:
a microfluidic channel;
a set of electrolysis electrodes positioned in an upstream portion of the microfluidic channel and configured to generate microbubbles from the induced sweat; and
a set of impedance sensing electrodes positioned in a downstream portion of the microfluidic channel and configured to detect the generated microbubbles.

5. The device of claim 4, wherein the set of impedance sensing electrodes includes a first set of impedance sensing electrodes positioned in the downstream portion of the microfluidic channel, and a second set of impedance sensing electrodes positioned in the downstream portion of the microfluidic channel and spaced apart from the first set of impedance sensing electrodes.

6. The device of claim 5, wherein the processor is configured to derive a time difference between two detection time points of the microbubbles at the first set of impedance sensing electrodes and the second set of impedance sensing electrodes, and to derive the secretion rate of the induced sweat based on the time difference.

7. A method for sweat analysis, comprising:
deriving a concentration of a target analyte in sweat;
deriving a secretion rate of the sweat; and
deriving a concentration of the target analyte in blood from the concentration of the target analyte in the sweat and the secretion rate.

8. The method of claim 7, wherein deriving the concentration of the target analyte in the blood is performed using a linear model relating the concentration of the target analyte in the sweat to the concentration of the target analyte in the blood.

9. The method of claim 7, wherein deriving the secretion rate of the sweat includes:
generating microbubbles from the sweat;
deriving a time difference between two detection time points of the microbubbles at a first set of impedance sensing electrodes and a second set of impedance sensing electrodes; and
deriving the secretion rate of the sweat based on the time difference.

10. A non-transitory computer-readable storage medium comprising instructions to:
derive a concentration of a target analyte in sweat;
derive a secretion rate of the sweat; and
derive a concentration of the target analyte in blood from the concentration of the target analyte in the sweat and the secretion rate.

11. The computer-readable storage medium of claim 10, wherein the instructions to derive the secretion rate of the sweat includes instructions to:
direct generation of microbubbles from the sweat;
derive a time difference between two detection time points of the microbubbles at a first set of impedance sensing electrodes and a second set of impedance sensing electrodes; and
derive the secretion rate of the sweat based on the time difference.
